(19) United States
(12) Patent Application Publication
(10) Pub. No.: US 2023/0406729 A1
(43) Pub. Date: Dec. 21, 2023

(54) METHOD FOR ANALYZING METAL IMPURITY CONTENT

(71) Applicant: ORGANO CORPORATION, Tokyo (JP)

(72) Inventor: Kyohei TSUTANO, Tokyo (JP)

(73) Assignee: ORGANO CORPORATION, Tokyo (JP)

(21) Appl. No.: 18/036,525

(22) PCT Filed: Oct. 13, 2021
(86) PCT No.: PCT/JP2021/037874

§ 371 Date: May 11, 2023

(30) Foreign Application Priority Data
Nov. 12, 2020 (JP) 2020-188471

(51) Int. Cl. C02F 1/42; G01N 33/18; G01N 30/96

(52) U.S. Cl. CPC C02F 1/42; G01N 33/1853; G01N 30/96

(57) ABSTRACT

A method for more accurately analyzing the content of a metal impurity in a liquid (ultrapure water) containing a low concentration of the metal impurity includes: passing the liquid through an ion exchanger; eluting and recovering the metal impurity captured in the ion exchanger with an eluent; and analyzing the eluent containing the eluted metal impurity and measuring the content of the metal impurity in the eluent, wherein the ion exchanger is used by connecting multiple ion exchangers of the same ion type in series, the volume of the porous ion exchangers per unit is 0.5 to 5.0 ml, and the differential pressure coefficient is 0.01 MPa/LV/m or less.

### TECHNICAL FIELD

The present invention relates to an analysis method for analyzing the content of metal impurities contained in a trace amount in a liquid such as ultrapure water, process water in an ultrapure water production process, a chemical used for cleaning semiconductors, or an organic solvent, and a measurement apparatus to be used in the method.

### BACKGROUND ART

In a semiconductor manufacturing process and a pharmaceutical manufacturing process, ultrapure water with extremely low content of ionic impurities is used. Therefore, in the production of ultrapure water used in the semiconductor manufacturing process and the pharmaceutical manufacturing process, it is important to determine the content of ionic impurities contained in trace amounts in the final produced ultrapure water or in the process water of the ultrapure water production process.

Patent Document 1 discloses an analysis method for passing a predetermined amount of fluid through a porous membrane having a functional group having an ion exchange function, causing impurities in the fluid to be trapped in the porous membrane, eluting the trapped impurities from the porous membrane, measuring the concentration of impurities in the elute, and calculating the concentration of impurities in the fluid from this measured concentration.

By the way, although the type and morphology of metal impurities in ultrapure water are not clear, they may exist as colloids in an aggregated state or fine particles in a dispersed state in addition to the form of ions. The surface charge density of colloids and fine particles is smaller than that of ions, and they have less electrostatic interaction with ion exchange resins.

Patent Document 2 discloses a method for analyzing a trace amount of metal impurities in ultrapure water by using a monolithic organic porous ion exchanger instead of the porous membrane.

The monolithic organic porous ion exchanger has a network-like flow path, and has an action of physically adsorbing or capturing fine particles in addition to an electrostatic interaction. Further, by using the monolithic organic porous anion exchanger, it is possible to adsorb or capture the metal impurities in the complexed anion state. Further, by using a monolithic organic porous cation exchanger, metal ions in a cation state can be adsorbed or captured. In other words, it can effectively adsorb or trap metallic impurities in ultrapure water.

### PRIOR ART DOCUMENTS

### Patent Documents

Patent Document 1: JP 2001-153854 A
Patent Document 2: WO 2019/221186 A1

### SUMMARY OF THE INVENTION

### Technical Problem

The analysis method described in Patent Document 1 enables analysis at a sub-μg/L level (sub-ppb level). Furthermore, in recent years, it has become necessary to analyze impurities having a lower concentration, such as impurities in ultrapure water.

The method described in Patent Document 2 includes: a step of passing water to be analyzed through a monolithic organic porous anion exchanger, thereby allowing the monolithic organic porous anion exchanger to capture metal impurities in the water to be analyzed; a step of passing an eluent through the monolithic organic porous anion exchanger which has been allowed to capture metal impurities in the water to be analyzed, to collect an effluent, thereby obtaining a collected eluent containing metal impurities in the water to be analyzed eluted from the monolithic organic porous anion exchanger; and a step of measuring a content of each metal impurity in the collected eluent. The method is capable of analyzing metal impurities at the ng/L (ppt) level. Further, there is also disclosed embodiments in which the monolithic organic porous anion exchanger is changed to a monolithic organic porous cation exchanger, and the anion exchanger and the cation exchanger are used in combination.

Alkali metals and alkaline earth metals are less likely to be adsorbed by anion exchangers, and boron and the like are less likely to be adsorbed by cation exchangers. Even if there is a difference in adsorption performance depending on the functional group of monolith, by using an anion exchanger and a cation exchanger in combination, almost complete adsorption of more than 99% is possible.

Here, the lower the concentration of the metal impurities to be analyzed, the more problematic is the influence of metal impurities contained outside the analysis target. Therefore, it is necessary to increase the amount of liquid passed through the system and increase the concentration ratio by ion exchange or other means. However, if the concentration is increased, ions may not be sufficiently adsorbed and captured by the ion exchanger and may leak out, resulting in inaccurate analysis of the metal impurity content in the liquid.

Accordingly, an object of the present invention is to provide a method for more accurately analyzing the content of metal impurities in a liquid containing low-concentration metal impurities.

### Means for Solving the Problem

The above object is solved by the present invention shown below.

That is, according to one aspect of the present invention, it is provided a method for analyzing a content of a metal impurity in a liquid, which includes:
a liquid passing step of passing the liquid through an ion exchanger;
an elution step of eluting and recovering the metal impurity captured in the ion exchanger with an eluent; and
a measurement step of analyzing the eluent containing the eluted metal impurity and measuring the content of the metal impurity in the eluent,
wherein the ion exchanger is used by connecting two or more units of the same ion-type ion exchanger in series,
the volume of the ion exchanger per unit is 0.5 to 5.0 ml, and the differential pressure coefficient per unit is 0.01 MPa/LV/m or less.

### Effect of the Invention

According to the present invention, it is possible to provide a method capable of more accurately analyzing the content of metal impurities of less than 1 ng/L in a liquid.

### BRIEF DESCRIPTION OF THE DRAWINGS

FIG. 1 It is a conceptual diagram explaining an example of the combination (measurement apparatus) of the ion exchanger of this invention.

FIG. 2 It is a conceptual diagram explaining another example of the combination of ion exchangers (measurement apparatus) of this invention.

FIG. 3 It is a conceptual diagram explaining another example of the combination of ion exchangers (measurement apparatus) of this invention.

### MODE FOR CARRYING OUT THE INVENTION

The analysis method of the present invention is a method for analyzing the content of metal impurities in a liquid, which includes:
a liquid passing step of passing the liquid through an ion exchanger;
an elution step of eluting and recovering the metal impurities captured in the ion exchanger with an eluent; and
a measurement step of analyzing the eluent containing the eluted metal impurities and measuring the content of the metal impurities in the eluent,
wherein the ion exchanger is used by connecting two or more units of the same ion-type ion exchanger in series, and the volume of the ion exchanger per unit is 0.5 to 5.0 ml.

In particular, in the present invention, the elution step and the measurement step are performed for each unit of the ion exchanger in order from the upper stage, and the content of metal impurities in the liquid measured in the measurement step is less than the lower limit of quantification. In this case, the total amount of the metal impurities in the liquid until it becomes less than the lower limit of quantification is defined as the content of the metal impurities in the liquid.

In the present invention, the ion exchanger used is not particularly limited, and can be either inorganic or organic, as long as a functional group having an ion exchange ability is introduced, such as a film-like, granular (resin), or porous material. It is especially preferable to use a porous ion exchanger described later, particularly preferable to use a monolithic organic porous ion exchanger. Hereinafter, a case where the monolithic organic porous ion exchanger (simply referred to as a monolith ion exchanger) is used will be described. Examples of the liquid to be analyzed include ultrapure water, process water in the ultrapure water production process, chemicals and organic solvents used for semiconductor cleaning, and other liquids in which the presence of a very small amount of metal impurities is a problem. Hereinafter, ultrapure water will be described as an example as a liquid.

(Liquid Passing Step)

The ultrapure water to be analyzed is passed through a porous ion exchanger (monolith ion exchanger), and metal impurities in the ultrapure water are captured by the monolith ion exchanger.

The ultrapure water to be analyzed in the present invention includes ultrapure water obtained by an ultrapure water production process for producing ultrapure water used in use points such as a semiconductor manufacturing process and a pharmaceutical manufacturing process, or process water during the ultrapure water production process. In the present invention, metal impurities of less than 1 ng/L contained in this ultrapure water are analyzed. Here, “less than 1 ng/L” is the concentration of metal impurities based on one metal element.

In the present invention, the process water during the ultrapure water production process is all water generated during the ultrapure water production process (the same applies hereinafter), for example, water transferred from the primary pure water production system to the secondary pure water production system in the ultrapure water production process, water transferred from the ultraviolet oxidizer of the secondary pure water production system to a non-regenerative cartridge polisher filled with an ion exchange resin, water transferred from the non-regenerative cartridge polisher filled with the ion exchange resin to a degassing membrane unit, water transferred from the degassing membrane unit to an ultrafiltration membrane device and water transferred from the ultrafiltration membrane device to the point of use.

The metal impurities to be analyzed are either one or two or more elements of Li, Be, B, Na, Mg, Al, K, Ca, Sc, Ti, V, Cr, Mn, Fe, Co, Ni, Cu, Zn, Ga, As, Sr, Zr, Mo, Pd, Ag, Cd, Sn, Ba, W, Au and Pb. In particular, alkali metal and alkaline earth metal elements are preferable.

In addition, fine particles may be contained in the ultrapure water used in the semiconductor manufacturing process. These fine particles include, for example, fine particles originally contained in source water, metal oxide fine particles generated from a piping material or a joint in a liquid feeding line of ultrapure water, and the like. Therefore, in ultrapure water used in the semiconductor manufacturing process, it is necessary to analyze the content of such fine particles in addition to the analysis of the content of ionic impurities. The size of the metal fine particles is not particularly limited, but is, for example, 1 to 100 nm.

Further, the metal impurities exist in the state of ionic impurities, fine particles such as colloidal or monodisperse, and complexes. Each ionic impurity element exists in the water to be analyzed in a state of a cation, a state of an oxo anion, or a mixed state of the cation and the oxo anion. Further, in the water to be analyzed, the metal impurity fine particles exist in a colloidal or monodisperse state.

The monolith ion exchanger is molded into a predetermined size and shape, sealed in a predetermined container, and connected in a plurality of series. The shape of the monolith ion exchanger should be a columnar structure, preferably cylindrical or prismatic (e.g., 3 to 8 prismatic columns).

The volume of the ion exchanger per unit is 0.5 to 5.0 ml and a differential pressure coefficient of 0.01 MPa/LV/m or less.

Further, the “1 unit” in the present invention is an ion exchanger enclosed in one container.

Such an ion exchanger is housed in a container having an inlet and an outlet for each unit, and “connected in series” means that the outlet and the downstream ion exchanger of the container containing the upstream ion exchanger are contained.

Further, “plurality” means connecting two or more containers, but the pressure loss tends to increase as the number of connections increases, and it is not necessary to connect an excessively large number of containers.

In the present invention, the upper limit of the number of connections cannot be unconditionally limited depending on the characteristics and size of the ion exchanger to be used, which will be described later, but the content of metal impurities analyzed based on the ion exchanger in the final stage is used. It is preferable to connect the minimum number that is less than the lower limit of quantification.

When a plurality of containers is connected in series, for example, the elution step (described later) and the measurement step (described later) are performed in order from the upper side (upstream side in the liquid flow direction) for each unit and the content of metal impurities in the liquid measured in the measurement step is less than the lower limit of quantification, the total content of metal impurities in the liquid until it becomes less than the lower limit of quantification can be taken as the content of metal impurities in the liquid.

If the lower limit of quantification is not reached in the lowermost ion exchanger (downstream in the flow direction of the liquid), an additional ion exchanger may be added to the downstream side of the lowermost ion exchanger, or it is desirable to reduce the concentration of the ion exchanger (the total flow rate of the ion exchanger).

In addition, the ion exchanger stored in one container may be referred to as “flow cell”.

The monolith ion exchanger according to the present invention is a porous material in which an ion exchange group (cation exchange group or anion exchange group) is introduced into a monolithic organic porous material. The monolithic organic porous material relating to the monolith ion exchanger is a porous material in which the skeleton is formed of an organic polymer and has a large number of connecting holes serving as liquid flow paths between the skeletons. The monolith ion exchanger is a porous material in which ion exchange groups are introduced into the skeleton of the monolithic organic porous material in uniform distribution.

In the present specification, the “monolithic organic porous material” is also simply referred to as “monolith”, and the “monolithic organic porous ion exchanger” in which ion exchange groups are introduced into the monolith is simply “monolith ion exchanger”. Further, a substance having an anion-exchange group introduced therein is referred to as an “anion-type monolith ion exchanger”, and a substance having a cation-exchange group introduced into the monolith is referred to as a “cation-type monolith ion exchanger”.

The monolith ion exchanger according to the present invention is obtained by introducing an ion exchange group into a monolith, and its structure is an organic porous material composed of a continuous skeleton phase and a continuous pore phase. The thickness of the continuous skeleton is preferably 1 to 100 μm, the average diameter of continuous pores is 1 to 1000 μm, and the total pore volume is preferably 0.5 to 50 mL/g.

The thickness of the continuous skeleton of the monolith ion exchanger in a dry state is preferably 1 to 100 μm. When the thickness of the continuous skeleton of the monolith ion exchanger is 1 μm or more, the ion exchange capacity per volume does not decrease, the decrease in mechanical strength is suppressed, and the deformation of the monolith ion exchanger can be particularly suppressed when the liquid is passed at a high flow velocity. On the other hand, if the thickness of the continuous skeleton of the monolith ion exchanger is 100 μm or less, the skeleton does not become too thick. The thickness of the continuous skeleton is determined by SEM observation.

The average diameter of the continuous pores of the monolith ion exchanger in a dry state is preferably 1 to 1000 μm. When the average diameter of the continuous pores of the monolith ion exchanger is 1 μm or more, it is possible to suppress an increase in pressure loss during water flow. On the other hand, when the average diameter of the continuous pores of the monolith ion exchanger is 1000 μm or less, the contact between the liquid to be treated and the monolith ion exchanger is sufficient, and a predetermined capturing force can be maintained. The average diameter of the continuous pores of the monolith ion exchanger in the dry state is measured by the mercury intrusion method and refers to the maximum value of the pore distribution curve obtained by the mercury intrusion method.

The total pore volume of the monolith ion exchanger in a dry state is preferably 0.5 to 50 mL/g. When the total pore volume of the monolith ion exchanger is 0.5 mL/g or more, the contact efficiency of the liquid to be treated can be sufficiently secured, and further, the amount of permeated liquid per unit cross-sectional area is not a problem, and the treatment amount can be suppressed. On the other hand, when the total pore volume of the monolith ion exchanger is 50 mL/g or less, a desired ion exchange capacity per volume can be secured and a predetermined capturing power can be maintained. In addition, the decrease in mechanical strength is suppressed, and it is possible to prevent the monolith ion exchanger from being significantly deformed, especially when the liquid is passed at high speed, and the pressure loss at the time of passing the liquid is suddenly increased. The total pore volume is measured by the mercury intrusion method.

Examples of the structure of such a monolith ion exchanger include the open cell structure disclosed in JP2002-306976A and JP2009-62512A, and JP2009-67982A. Examples thereof include a co-continuous structure, a particle-aggregated structure disclosed in JP2009-7550A, and a particle-composite-type structure disclosed in JP 2009-108294A.

The ion exchange capacity per volume of the monolith ion exchanger is preferably 0.2 to 1.0 mg-equivalent/mL (water-wet state). When the ion exchange capacity of the monolith ion exchanger is 0.2 mg-equivalent/mL or more, the volume of treated water until breakthrough can be sufficiently secured as the volume of treated water per treatment of the present invention. On the other hand, if the ion exchange capacity is 1.0 mg-equivalent/mL or less, the pressure loss during water flow will be within the range where there is no problem. The ion exchange capacity of the porous body in which the ion exchange group is introduced only on the surface of the skeleton cannot be unconditionally determined depending on the type of the porous body or the ion exchange group, but is at most 500 μg-equivalent/g.

### <Elution Step>

In the present invention, the next step is to collect the metal impurities captured in the porous ion exchanger (monolith ion exchanger) by eluting them with an eluent. This step is called the “elution step”.

The eluent is an aqueous solution containing an acid. The acid contained in the eluent is not particularly limited as long as it does not affect the ion exchanger, and examples thereof include inorganic acids such as nitric acid, sulfuric acid, hydrochloric acid and phosphoric acid, and organic acids such as methane sulfonic acid. Of these, as the acid contained in the eluent, nitric acid, sulfuric acid, and hydrochloric acid are preferable because ionic impurity elements can be easily eluted from the monolith ion exchanger and a high-purity reagent is required.

The acid concentration in the eluent is not particularly limited, but the analysis method of the present invention can lower the acid concentration in the eluent, so that the lower limit of quantification can be lowered. Therefore, the acid concentration in the eluent is preferably 0.1 to 2.0N, more preferably 0.5 to 2.0N in that the lower limit of quantification is lowered. When the acid concentration is 0.1N or more, it is possible to suppress an increase in the amount of liquid to be recovered. On the other hand, when the acid concentration is 2.0N or less, it is possible to suppress an increase in the lower limit of quantification of the analyzer. The eluent is preferably one having a content of each metal impurity of 100 ppt or less, more preferably nitric acid or hydrochloric acid having a content of each metal impurity of 100 ppt or less, and nitric acid or hydrochloric acid, which has a content of each metal impurity of 10 ppt or less, is particularly preferred.

In the elution step, the volume of eluent to be passed through the monolith ion exchanger is appropriately selected depending on the type and thickness of the monolith ion exchanger, the water flow rate, and the like. Since the metal element is easily eluted from the monolith ion exchanger in the analysis method of the present invention, the metal impurity analysis method of the present invention can reduce the flowing volume of eluent. Then, the decrease in the flowing volume of the eluent leads to a reduction in the measurement time.

The conditions under which the eluent is passed through the monolith ion exchanger during the elution step are not restricted. The liquid passing speed expressed by the space velocity (SV) is preferably 20000 h⁻¹ or less, more preferably 10 to 4000 h⁻¹, and particularly preferably 300 to 1000 h⁻¹. The liquid passing speed represented by the linear velocity (LV) is preferably 1000 m/h or less, and particularly preferably 500 m/h or less. The liquid passing time is appropriately selected depending on the total liquid passing volume of the eluent and the liquid passing speed.

In the elution step, the metal impurities to be analyzed trapped in the monolith ion exchanger are eluted by the eluent and transferred into the eluent. Then, by performing the elution step, a recovered eluent containing the metal impurities to be analyzed is obtained.

### <Analysis/Measurement Step>

Next, a measurement step is performed to analyze the eluent containing the eluted metal impurities and to measure the content of the metal impurities in the eluent.

The method for measuring the content of each metal impurity in the recovered eluent is not particularly limited, and include methods using a Inductively Coupled Plasma Mass Spectrometry (ICP-MS), a Inductively Coupled Plasma Atomic Emission Spectroscopy (ICP-AES), an atomic absorption spectrophotometer, an ion chromatograph analyzer, etc. The measurement conditions are appropriately selected.

In the analysis method of the present invention, the type and content of each metal impurity in the recovered eluent obtained by performing the measurement step are determined. The content of each metal impurity in the ultrapure water to be analyzed is obtained from the recovered volume of the recovered eluent and the total volume of ultrapure water passed through the monolith ion exchanger in the ultrapure water passing step.

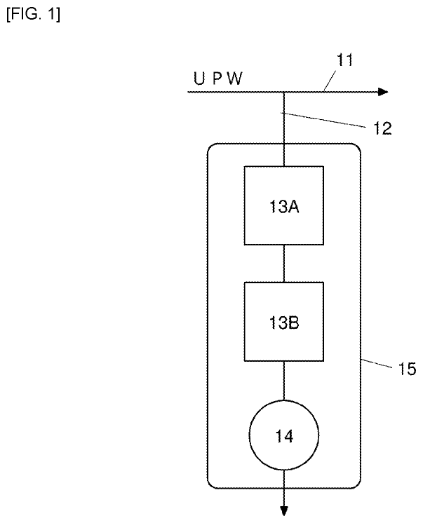

Examples of embodiments of the analysis method of the present invention will be described. For example, as shown in FIG. 1, in the ultrapure water production process in which the ultrapure water (UPW) obtained by the ultrapure water production unit (not shown) is supplied to the use point, a discharge pipe 12 for water to be analyzed is connected to the middle of a transfer pipe 11 for transferring ultrapure water to the use point. The other end of the discharge pipe 12 for water to be analyzed is connected to an inlet of a measurement apparatus 15 in which flow cells 13A and 13B equipped with monolith ion exchangers are connected in series and the integrated flow meter 14 is installed downstream thereof. Here, the monolith ion exchangers provided in the two flow cells have the same ionic form, and when the cationic monolith ion exchanger is installed in the flow cell 13A, the flow cell 13B is also set up cationic monolith ion exchange.

Next, after passing a predetermined volume of ultrapure water, the measurement apparatus 15 is removed from the discharge pipe 12 for water to be analyzed. At this time, the inside of the measurement apparatus 15 is removed by a method that does not cause impurities from being mixed from the outside, and the inside is sealed. Next, the flow cells 13A and 13B removed from the measurement apparatus 15 are attached to an elution device provided at a place different from the place where the ultrapure water production process is performed. The elution step is performed in which nitric acid or hydrochloric acid is passed through the eluent supply pipes of the elution device to the flow cells 13A and 13B, respectively, and metal impurities are eluted with the eluent and recovered. Next, the measurement step of measuring the content of metal impurities in the recovered eluent is performed. As described in WO 2019/221186A1, an eluent introduction pipe (not shown) for passing eluent may be arranged on an extraction pipe 12 for water to be analyzed or first and second branch pipes (16, 16′) described later, or to the measurement apparatus 15 itself. Thereby, the eluent can be passed through the flow cell with the measurement apparatus 15 (flow cell) attached to the ultrapure water production device to perform the elution step, and the content of metal impurities in the recovered eluent can be measured.

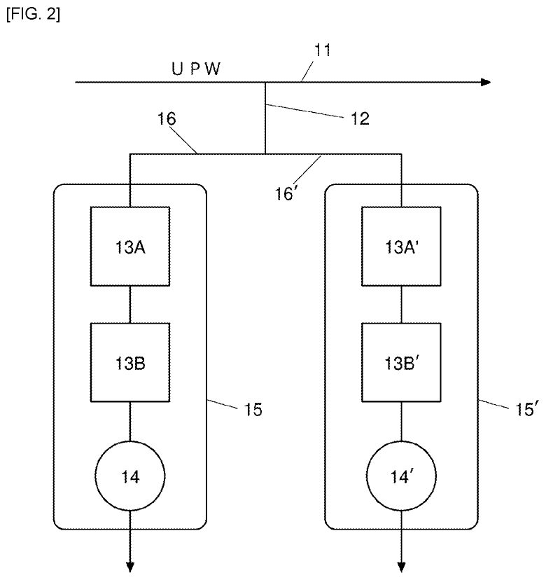

Other embodiments of the present invention will be described. For example, as shown in FIG. 2, in the ultrapure water production process in which the ultrapure water (UPW) obtained by the ultrapure water production device (not shown) is supplied to the use point, extraction pipe 12 for water to be analyzed is connected to the middle of the ultrapure-water-transfer pipe 11 for transferring the ultrapure water to the use point, and the other end of the extraction pipe 12 for water to be analyzed is branched into a first branch pipe 16 and a second branch pipe 16′. Then, the first branch pipe 16 is connected to the inlet of the measurement apparatus 15 in which flow cells 13A and 13B having a monolith ion exchanger, for example, a cationic ion exchanger, are connected in series and an integrated flow meter 14 is installed downstream thereof. Similarly, the second branch pipe 16′ is connected to the inlet of the measurement apparatus 15′ in which flow cells 13A′ and 13B′ having a monolith ion exchanger, for example, an anionic ion exchanger, are connected in series and an integrated flow meter 14′ is installed downstream thereof. At this time, the total volume of ultrapure water passed through the measurement apparatus 15 and 15′ is measured by the integrated flow meters 14 and 14′. After that, the elution step and the measurement step are carried out in the same manner. In this example, monolith ion exchangers of the same ionic form are connected in series, and monolith ion exchangers of different ionic form are connected in parallel.

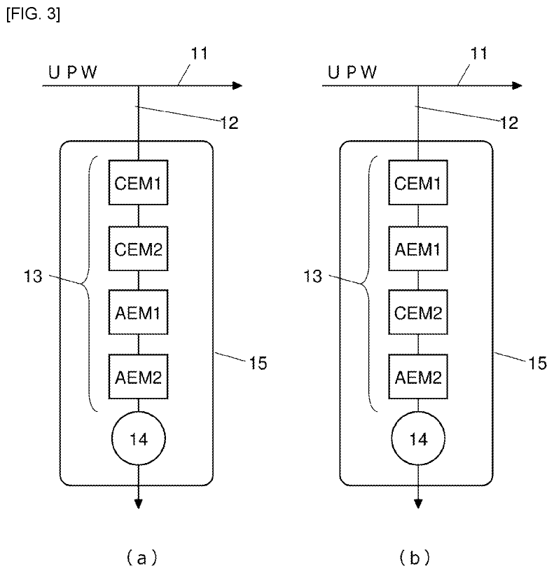

In further another embodiment, the cationic monolith ion exchanger and the anionic monolith ion exchanger can be connected and used in series. FIG. 3 shows a configuration example of a measurement apparatus when a cationic monolith ion exchanger (CEM) and an anionic monolith ion exchanger (AEM) are connected in series and used. As the order, any of CEM1→CEM2→AEM1→AEM2 shown in FIG. 3(a) and CEM1→AEM1→CEM2→AEM2 shown in FIG. 3(b) is possible. In particular, as shown in FIG. 3(b), the monolith ion exchangers having the same ion form are connected in series, even if they are not contiguous. Further, the CEM and AEM may be enclosed in one flow cell for use. In that case, the number is two instead of four as shown in the figure. The order of the cationic form and the anionic form is not particularly limited, and may be an order other than that shown in FIG. 3. Further, in the example of FIG. 3, two (2 units) monolith ion exchangers are connected in series for each ion form, but depending on the metal impurities contained in the ultrapure water, 2 units of the cationic monolith ion exchanger and 1 unit of an anionic monolith ion exchanger, for a total of 3 units, may be used. In particular, in the present invention, since leakage of cationic species is likely to occur, it is preferable to connect at least two units of a cationic monolith ion exchanger in series.

Further, FIGS. 1 to 3 show an example in which two units of monolith ion exchangers of the same ion form are connected in series, but the present invention is not limited to these, and three or more units may be connected as described above.

In the monolith ion exchanger, the introduced ion-exchange groups are uniformly distributed not only on the surface of the monolith but also inside the skeleton of the monolith. The term “ion-exchange groups are uniformly distributed” as used herein means that the distribution of ion-exchange groups is uniformly distributed on the surface and inside the skeleton on at least the μm order. The distribution of ion-exchange groups can be easily confirmed by using an Electron Probe Micro Analyzer (EPMA). Further, if the ion-exchange groups are uniformly distributed not only on the surface of the monolith but also inside the skeleton of the monolith, the physical and chemical properties of the surface and the inside of the monolith can be made uniform, so that the durability against swelling and shrinkage can be improved.

Examples of the cation-exchange group introduced into the cationic monolith ion exchanger include a sulfonic acid group, a carboxyl group, an iminodiacetic acid group, a phosphoric acid group, and a phosphoric acid ester group.

Examples of the anion-exchange group introduced into the anionic monolith ion exchanger include quaternary ammonium groups such as a trimethylammonium group, a triethylammonium group, a tributyl-ammonium group, a dimethyl-hydroxyethyl-ammonium group, a dimethyl-hydroxypropyl-ammonium group and a methyl-dihydroxyethyl-ammonium group, a tertiary sulfonium group and a phosphonium group.

In the monolith ion exchanger, the material constituting the continuous skeleton is an organic polymer material having a crosslinked structure. The cross-linking density of the polymer material is not particularly limited, but contains 0.1 to 30 mol %, preferably 0.1 to 20 mol % of the cross-linked structural units with respect to all the structural units constituting the polymer material. When the crosslinked structural unit is 0.1 mol % or more, the mechanical strength is not insufficient, while when it is 30 mol % or less, the introduction of the ion exchange group is not difficult. The kind of the polymer material is not particularly limited, and includes crosslinkable polymers, for example, an aromatic vinyl polymer such as polystyrene, poly(α-methylstyrene), polyvinyltoluene, polyvinylbenzyl chloride, polyvinylbiphenyl, polyvinylnaphthalene; a polyolefin such as polyethylene and polypropylene; poly(halogenated polyolefin) such as polyvinylchloride and polytetrafluoroethylene; nitrile polymer such as polyacrylonitrile; (meth)acrylic polymer such as methyl polymethacrylate, glycidylpolymethacrylate, polyethylacrylate and the like. The above polymer may be a polymer obtained by copolymerizing a single vinyl monomer and a cross-linking agent, a polymer obtained by polymerizing a plurality of vinyl monomers and a cross-linking agent, or a blend of two or more kinds of polymers. Among these organic polymer materials, cross-linked polymers of aromatic vinyl polymers are preferred due to their ease of continuous structure formation, ease of introducing ion-exchange groups, high mechanical strength, and high stability against acids or alkalis. Especially, styrene-divinylbenzene copolymer and vinylbenzyl chloride-divinylbenzene copolymer are mentioned as preferable materials.

### Embodiments of Monolith Ion Exchanger

Embodiments of the monolith ion exchanger include a first monolith ion exchanger and a second monolith ion exchanger shown below. In addition, embodiments of the monolith into which the ion-exchange group is introduced include a first monolith and a second monolith shown below.

### <Explanation of the First Monolith and the First Monolith Ion Exchanger>

The first monolith ion exchanger has a continuous pore structure with interconnected macropores and common openings (mesopores) with an average diameter of 1 to 1000 μm in a dry state within the walls of the macropores. It has the total pore volume of 1 to 50 mL/g in the dry state, it has ion exchange groups, and the ion exchange groups are uniformly distributed, per volume. The ion exchange capacity per volume of the monolith ion exchanger is 0.1 to 1.0 mg equivalent/mL (water-wet state). The first monolith is a monolith before the introduction of the ion exchange group, and has a continuous pore structure with interconnected macropores and common openings (mesopores) with an average diameter of 1 to 1000 μm in a dry state within the walls of the macropores. It has the total pore volume of 1 to 50 mL/g in the dry state.

The first monolith ion exchanger is a continuous macropore structure in which bubble-like macropores overlap each other, and the overlapping portion has a common opening (mesopore) having an average diameter of 1 to 1000 μm, preferably 10 to 200 μm, particularly preferably 20 to 100 μm in a dry state, most of which are open pore structures. In the open pore structure, when a liquid is flowed, a flow path becomes a flow path in a cavity formed by the macropore and the mesopore. The overlap of macropores is 1 to 12 macropores per one macropore, and the overlap for most macropores is 3 to 10 macropores per one macropore. When the average diameter of the mesopore in the dry state is 1 μm or more, the diffusivity of the liquid to be treated into the inside of the monolith ion exchanger does not decrease, and when the average diameter of the mesopore in the dry state is 1000 μm or less, the monolith ion exchanger sufficiently contacts with the liquid to be treated. Since the structure of the first monolith ion exchanger is the continuous macropore structure as described above, a group of macropores and a group of mesopores can be uniformly formed, and the pore volume and specific surface area can be significantly increased as compared with the particle aggregation type porous body as described in JP 8-252579A or the like can be formed.

In the present invention, the average diameter of the opening of the first monolith in the dry state and the average diameter of the opening of the first monolith ion exchanger in the dry state are measured by the mercury intrusion method and refers to the maximum value of the pore distribution curve obtained by the mercury intrusion method.

The total pore volume per weight of the first monolith ion exchanger in the dry state is 1 to 50 mL/g, preferably 2 to 30 mL/g. When the total pore volume is 1 mL/g or more, the contact efficiency of the liquid to be treated does not decrease, the permeation volume per unit cross-sectional area becomes sufficient, and the decrease in processing capacity can be suppressed. On the other hand, when the total pore volume is 50 mL/g or less, sufficient mechanical strength can be obtained, and it is possible to suppress the large deformation of the monolith ion exchanger particularly when the liquid is passed at a high flow rate. Further, the contact efficiency between the liquid to be treated and the monolith ion exchanger is sufficiently satisfied, and there is no problem of catchability. Since the total pore volume is at most 0.1 to 0.9 ml/g in the conventional particulate porous ion exchange resin, the high pore volume of 1 to 50 ml/g and high specific surface area, which are higher than that in the conventional one.

In the first monolith ion exchanger, the material constituting the skeleton is an organic polymer material having a crosslinked structure. The cross-linking density of the polymer material is not particularly limited, but contains 0.3 to 10 mol %, preferably 0.3 to 5 mol % of crosslinked structural units with respect to all the structural units constituting the polymer material. When the crosslinked structural unit is 0.3 mol % or more, the mechanical strength is sufficient, while when it is 10 mol % or less, the introduction of the ion exchange group is not hindered.

The kinds of the organic polymer materials constituting the skeleton of the first monolith ion exchanger are not particularly limited, and for example, include crosslinked polymers of aromatic polymers such as polystyrene, poly(α-methylstyrene), polyvinyltoluene, polyvinylbenzyl chloride, polyvinylbiphenyl, and polyvinylnaphthalene; polyolefins such as polyethylene and polypropylene; poly(halogenated-polyolefins) such as polyvinyl chloride and polytetrafluoroethylene; nitrile-based polymers such as polyacrylonitrile; and (meth)acrylic polymers such as methyl polymethacrylate, glycidyl polymethacrylate, ethyl polyacrylate. The organic polymer may be a polymer obtained by copolymerizing a single vinyl monomer and a cross-linking agent, or a polymer obtained by polymerizing a plurality of vinyl monomers and a cross-linking agent, and two or more kinds of polymers may be blended. Among these organic polymer materials, the crosslinked polymers of aromatic vinyl polymers are preferable due to the ease of forming a continuous macropore structure, the ease of introducing ion-exchange groups, the high mechanical strength, and the high stability against acids or alkalis. Particularly, styrene-divinylbenzene copolymer and vinylbenzyl chloride-divinylbenzene copolymer are mentioned as the more preferable material.

As the ion exchange group introduced into the first monolith ion exchanger, the above ion exchange group can be mentioned. The same applies to the second monolith ion exchanger.

In the first monolith ion exchanger (the same applies to the second monolith ion exchanger), the introduced ion exchange groups are uniformly distributed not only on the surface of the porous body but also inside the skeleton of the porous body. The distribution of ion exchange groups is confirmed by using EPMA as described above. In addition, such uniform distribution of ion exchange groups allows uniform physical and chemical properties on the surface and inside, thus improving durability against swelling and shrinkage.

The ion exchange capacity per volume of the first monolith ion exchanger is 0.1 to 1.0 mg-equivalent/mL (water-moistened state). When the ion exchange capacity per volume in a water-wet state is within the above range, the removal performance is high and the life is extended. The ion exchange capacity of the porous body in which the ion exchange group is introduced only on the surface cannot be unconditionally determined depending on the type of the porous body or the ion exchange group, but is at most 500 μg-equivalent/g.

### <Method for Producing the First Monolith and the First Monolith Ion Exchanger>

The method for producing the first monolith is not particularly limited, but will be described below an example of a production method according to the method described in JP 2002-306976A. That is, the first monolith is obtained by mixing an oil-soluble monomer containing no ion exchange group, a surfactant, water and, if necessary, a polymerization initiator to obtain a water-in-oil emulsion, which is then polymerized to form a monolith. This method for producing the first monolith is preferable in that the porous structure of the monolith can be easily controlled.

The oil-soluble monomer containing no ion exchange group used in the production of the first monolith does not contain any of a cation exchange group such as a carboxylic acid group and a sulfonic acid group and an anion exchange group such as a quaternary ammonium group. It refers to a monomer that has low solubility in water and is lipophilic. Specific examples of these monomers include styrene, α-methylstyrene, vinyltoluene, vinylbenzyl chloride, divinylbenzene, ethylene, propylene, isobutene, butadiene, isoprene, chloroprene, vinyl chloride, vinyl bromide, vinylidene chloride, tetrafluoroethylene, acrylonitrile, methacrylonitrile, vinyl acetate, methyl acrylate, ethyl acrylate, butyl acrylate, 2-ethylhexyl acrylate, trimethylolpropane triacrylate, butanediol diacrylate, methyl methacrylate, ethyl methacrylate, propyl methacrylate, butyl methacrylate, 2-ethylhexyl methacrylate, cyclohexyl methacrylate, benzyl methacrylate, glycidyl methacrylate, ethylene glycol dimethacrylate and the like. These monomers may be used alone or in combination of two or more. However, in the present invention, a crosslinkable monomer such as divinylbenzene or ethylene glycol dimethacrylate is selected as at least one component of the oil-soluble monomer. The content thereof is preferably 0.3 to 10 mol % in the total oil-soluble monomer, preferably 0.3 to 5 mol %, because the ion exchange group can be quantitatively introduced in a later step and a practically sufficient mechanical strength can be secured.

The surfactant used in the production of the first monolith is, but not particularly limited, capable of forming a water-in-oil (W/O) emulsion when water is mixed with an oil-soluble monomer containing no ion exchange group. Examples thereof include nonionic surfactants such as sorbitan monooleate, sorbitan monolaurate, sorbitan monopalmitate, sorbitan monostearate, sorbitan trioleate, polyoxyethylene nonylphenyl ether, polyoxyethylene stearyl ether, polyoxyethylene sorbitan monooleate; anionic surfactants such as potassium oleate, sodium dodecylbenzenesulfonate, sodium dioctyl sulfosuccinate; cationic surfactants such as distearyl dimethylammonium chloride; and amphoteric surfactants such as lauryldimethylbetaine. These surfactants can be used alone or in combination of two or more thereof. The water-in-oil emulsion is an emulsion in which the oil phase is a continuous phase and water droplets are dispersed therein. The amount of the above-mentioned surfactant added varies greatly depending on the kind of the oil-soluble monomer and the size of the target emulsion particles (macropores), and therefore cannot be unequivocally determined, but it can be selected in the range of about 2 to 70% by mass of the total amount of the oil-soluble monomer and the surfactant. In addition, although not always essential, in order to control the shape and size of the cavity of the monolith, alcohols such as methanol and stearyl alcohol; carboxylic acids such as stearic acid; hydrocarbons such as octane, dodecane and toluene; cyclic ether such as tetrahydrofuran and dioxane can also coexist in the system.

Further, in the production of the first monolith, a compound that generates radicals by heat and light irradiation is preferably used as the polymerization initiator used as necessary when forming the monolith by polymerization. The polymerization initiator may be water-soluble or oil-soluble, and includes, for example, azobisisobutyronitrile, azobisdimethylvaleronitrile, azobiscyclohexanenitrile, azobiscyclohexanecarbonitrile, benzoyl peroxide, potassium persulfate, ammonium persulfate, hydrogen peroxide-ferrous chloride, sodium persulfate-sodium acid sulfite, tetramethylthium disulfide and the like. However, in some cases, the polymerization proceeds only by heating or light irradiation without adding the polymerization initiator, so that it is not necessary to add the polymerization initiator in such a system.

In the production of the first monolith, there is no particular limitation on the mixing method to form a water droplet emulsion in oil by mixing the oil-soluble monomer containing no ion exchange group, the surfactant, water and the polymerization initiator, the method can be used a method of mixing each component at once, or a method of mixing oil-soluble components, which are an oil-soluble monomer, the surfactant, and an oil-soluble polymerization initiator, and a water-soluble component, which is water or a water-soluble polymerization initiator, are separately dissolved, and then uniformly mixing respective components. There is no particular limitation on the mixing device for forming the emulsion, and a normal mixer, homogenizer, high-pressure homogenizer, or so-called planetary agitator in which materials to be treated are placed in a mixing container, and the mixing container is rotated around a revolution axis in an inclined state, thereby agitating and mixing the materials to be treated can be used, and an appropriate device may be selected to obtain the desired emulsion particle size. Further, the mixing conditions are not particularly limited, and the stirring rotation speed and the stirring time capable of obtaining the desired emulsion particle size can be arbitrarily set. Among these mixing devices, the planetary agitator is preferably used because it can uniformly generate water droplets in the W/O emulsion and the average diameter thereof can be arbitrarily set in a wide range.

In the production of the first monolith, various conditions can be selected for the polymerization conditions to polymerize the water-in-oil droplet emulsion thus obtained, depending on the kind of the monomer and the initiator system. For example, when azobisisobutyronitrile, benzoyl peroxide, potassium persulfate or the like is used as the polymerization initiator, it can be polymerized by heating at 30 to 100° C. for 1 to 48 hours in a sealed container under an inert atmosphere. When hydrogen peroxide-ferrous chloride, sodium persulfate-sodium acid sulfite, etc. are used as the initiator, the polymerization can be carried out at 0 to 30° C. for 1 to 48 hours in a sealed container under an inert atmosphere. After completion of the polymerization, the inner contents are taken out and Soxhlet extracted with a solvent such as isopropanol to remove the unreacted monomer and the residual surfactant to obtain the first monolith.

The method for producing the first monolith ion exchanger is not particularly limited, and in the method for producing the first monolith, examples thereof include a method of polymerizing a monomer containing an ion exchange group instead of the monomer having no ion exchange group in the above production of the first monolith to form a monolithic anion exchanger in one step, for example, a monomer having an anion exchange group such as a monomethylammonium, a dimethylammonium group, or a trimethylammonium group by introducing to the oil-soluble monomer having no ion exchange group, or a method of forming a first monolith by polymerizing using a monomer having no ion exchange group and then introducing an ion exchange group. Among these methods, the method of forming a first monolith by polymerizing using a monomer having no ion exchange group and then introducing an ion exchange group is preferable because the control of the porous structure of the monolith ion exchanger is easy and it is possible to quantitatively introduce an ion exchange group.

The method for introducing an ion exchange group into the first monolith is not particularly limited, and known methods such as polymer reaction and graft polymerization can be used. For example, as a method for introducing a quaternary ammonium group, if the monolith is a styrene-divinylbenzene copolymer or the like, a method of introducing a chloromethyl group by using a chloromethylmethyl ether or the like and then reacting with a tertiary amine; a method of producing by copolymerization of chloromethylstyrene and divinylbenzene and then reacting with a tertiary amine; a method of graft polymerization of N,N,N-trimethylammonium or N,N,N-trimethylammonium propylacrylamide by uniformly introducing a radical initiation group or a chain transfer group into a monolith on the surface of the skeleton and inside the skeleton; and a method of graft-polymerizing ethyl acrylate; similarly, and then introducing a quaternary ammonium group by functional group conversion can be mentioned. Among these methods, as a method for introducing a quaternary ammonium group, the method of introducing a chloromethyl group into a styrene-divinylbenzene copolymer with chloromethylmethyl ether or the like and then reacting with a tertiary amine, or the method of producing a monolith by copolymerization of chloromethylstyrene and divinylbenzene and then reacting with a tertiary amine is preferable in that an ion exchange group can be introduced uniformly and quantitatively. The ion exchange group to be introduced includes a quaternary ammonium group such as a trimethylammonium group, a triethylammonium group, a tributylammonium group, a dimethylhydroxyethylammonium group, a dimethylhydroxypropylammonium group and a methyldihydroxyethylammonium group, and a tertiary sulfonium groups, phosphonium groups and the like.

### <Explanation of the Second Monolith and the Second Monolith Ion Exchanger>

The second monolith ion exchanger is made of an aromatic vinyl polymer containing 0.1 to 5.0 mol % of crosslinked structural units in all the structural units. The aromatic vinyl polymer has a co-continuous structure composed of a three-dimensionally continuous skeleton having an average thickness of 1 to 60 μm in a dry state and three-dimensionally continuous pores having an average diameter of 10 to 200 μm between the skeletons. The second monolith ion exchanger has 0.5 to 10 mL/g of the total pore volume in the dry state, has an ion exchange group, and has 0.2 to 1.0 mg-equivalent/mL of the ion exchange capacity per volume (water-wet state), and is a monolith ion exchanger in which ion exchange groups are uniformly distributed in the monolith ion exchanger. The second monolith is a monolith before the introduction of the ion exchange group, and consists of an aromatic vinyl polymer having an average thickness of 0.1 to 5.0 mol % of crosslinked structural units in all the structural units, which is a co-continuous structure composed of a three-dimensionally continuous skeleton having an average thickness of 1 to 60 μm in a dry state and three-dimensionally continuous pores having an average diameter of 10 to 200 μm between the skeletons. It is an organic porous body having a total pore volume of 0.5 to mL/g in a dry state.

The second monolith ion exchanger is a co-continuous structural body composed of a three-dimensionally continuous skeleton having an average thickness of 1 to 60 μm, preferably 3 to 58 μm in a dry state, and a three-dimensionally continuous pores having an average diameter of 10 to 200 μm between the skeletons in a dry state, preferably 15 to 180 μm, particularly preferably 20 to 150 μm.

When the average diameter of the three-dimensionally continuous pores is 10 μm or more in the dry state, the liquid to be treated easily diffuses, and when it is 200 μm or less, the contact between the liquid to be treated and the monolith ion exchanger becomes sufficient, and as a result, the removal performance is sufficient. Further, when the average thickness of the skeleton is 1 μm or more in a dry state, the ion exchange capacity per volume does not decrease, and the decrease in mechanical strength is suppressed. Further, a capturing performance can be sufficiently obtained without lowering the contact efficiency between the reaction solution and the monolith ion exchanger. On the other hand, when the thickness of the skeleton is 60 μm or less, the skeleton does not become too thick and the diffusion of the liquid to be treated becomes uniform.

The average diameter of the opening of the second monolith in the dry state, the average diameter of the opening of the second monolith ion exchanger in the dry state, and the average diameter of the opening of a second monolith intermediate in the dry state obtained in step I of the production of the second monolith described below are determined by the mercury intrusion method and refers to the maximum value of the pore distribution curve obtained by the mercury intrusion method. Further, the average thickness of the skeleton of the second monolith ion exchanger in the dry state can be obtained by SEM observation of the second monolith ion exchanger in the dry state. Specifically, the SEM observation of the second monolith ion exchanger in the dry state is performed at least three times, each thickness of the skeleton in the obtained image is measured, and the average value thereof is taken as the average thickness. The skeleton is rod-shaped and has a circular cross-sectional shape, but may include a skeleton having a different diameter such as an elliptical cross-sectional shape. The thickness in this case is the average of the minor axis and the major axis.

The total pore volume per weight of the second monolith ion exchanger in a dry state is 0.5 to 10 mL/g. When the total pore volume is mL/g or more, the contact efficiency with the liquid to be treated can be ensured, and the volume of permeated liquid per unit cross-sectional area is not a problem, and the decrease in the treated amount is suppressed. On the other hand, when the total pore volume is 10 ml/g or less, the contact efficiency between the liquid to be treated and the monolith ion exchanger does not decrease, and the decrease in the capturing performance is suppressed. When the size of the three-dimensionally continuous pores and the total pore volume are within the above ranges, the contact with the liquid to be treated is extremely uniform and the contact area is also large.

In the second monolith ion exchanger, the material constituting the skeleton is an aromatic vinyl polymer containing 0.1 to 5 mol %, preferably 0.5 to 3.0 mol % of crosslinked structural units in the total structural units and is hydrophobic. When the crosslinked structural unit is 0.1 mol % or more, the mechanical strength may not be insufficient, while when it is 5 mol % or less, the structure of the porous body is less likely to deviate from the co-continuous structure. The kinds of aromatic vinyl polymer are not particularly limited, and examples thereof include polystyrene, poly (α-methylstyrene), polyvinyltoluene, polyvinylbenzyl chloride, polyvinylbiphenyl, and polyvinylnaphthalene. The polymer may be a polymer obtained by copolymerizing a single vinyl monomer and a cross-linking agent, or a polymer obtained by polymerizing a plurality of vinyl monomers and a cross-linking agent, and two or more kinds of polymers may be blended. Among these organic polymer materials, a styrene-divinylbenzene copolymer and vinylbenzyl chloride-divinylbenzene copolymer are preferable due to the ease of forming a co-continuous structure, the ease of introducing ion exchange groups, the high mechanical strength, and the due to the high stability against acid or alkalis.

The ion exchange group introduced into the second monolith ion exchanger is the same as the ion exchange group introduced into the first monolith ion exchanger.

In the second monolith ion exchanger, the introduced ion exchange groups are uniformly distributed not only on the surface of the porous body but also inside the skeleton of the porous body.

The second monolith ion exchanger has an ion exchange capacity of 0.2-1.0 mg-equivalent/mL (water-moistened state) per volume. Since the second monolith ion exchanger has high continuity and uniformity of three-dimensionally continuous pores, the substrate and the solvent diffuse uniformly. Therefore, the reaction progresses quickly. When the ion exchange capacity is in the above range, the removal performance is high and the life is extended.

### <Method for Producing the Second Monolith and the Second Monolith Ion Exchanger>

The second monolith is obtained by carrying out step I in which a water-in-oil emulsion is preparing by stirring a mixture of an oil-soluble monomer having no ion-exchange group, a surfactant, and water, then, the water-in-oil emulsion is polymerized to form a monolithic organic porous intermediate having a total pore volume of more than 16 mL/g and 30 mL/g or less of a continuous macropore structure (hereinafter, also referred to as monolith intermediate); step II in which a mixture of an aromatic vinyl monomer, 0.3-5 mol % of cross-linking agent in total of oil-soluble monomers having at least two vinyl groups in one molecule, an organic solvent that dissolves the aromatic vinyl monomer and the cross-linking agent but does not dissolve a polymer generated by polymerizing the aromatic vinyl monomer, and a polymerization initiator; and step III in which it is obtained by carrying out polymerization in the presence of the obtained monolithic intermediate in the step II to obtain a second monolith which is an organic porous body having a co-continuous structure.

In the step I relating to the method for producing the second monolith, the step I for obtaining a monolith intermediate may be carried out in accordance with the method described in JP 2002-306976A.

That is, in the step I according to the method for producing the second monolith, as the oil-soluble monomer containing no ion exchange group, it can be mentioned as a lipophilic monomer in which an ion exchange group such as a carboxylic acid group, a sulfonic acid group, a tertiary amino group, a quaternary ammonium group and the like does not contain, and being low-solubility in water. Specific examples of these monomers include aromatic vinyl monomers such as styrene, α-methylstyrene, vinyltoluene, vinylbenzyl chloride, vinylbiphenyl and vinylnaphthalene; α-olefins such as ethylene, propylene, 1-butene and isobutene; diene-based monomers such as butadiene, isoprene and chloroprene; halogenated olefins such as vinyl chloride, vinyl bromide, vinylidene chloride and tetrafluoroethylene; nitrile-based monomers such as acrylonitrile and methacrylic acid; vinyl esters such as vinyl acetate and vinyl propionate; (meth)acrylic polymers such as methyl acrylate, ethyl acrylate, butyl acrylate, 2-ethylhexyl acrylate, methyl methacrylate, ethyl methacrylate, propyl methacrylate, butyl methacrylate, 2-ethylhexyl methacrylate, cyclohexyl methacrylate, benzyl methacrylate, and glycidyl methacrylate. Among these monomers, preferred ones are aromatic vinyl monomers, and examples thereof include styrene, α-methylstyrene, vinyltoluene, vinylbenzyl chloride, and divinylbenzene. These monomers may be used alone or in combination of two or more. Here, it is preferable that a crosslinkable monomer such as divinylbenzene or ethylene glycol dimethacrylate is selected as at least one component of the oil-soluble monomer, and the content thereof is 0.3 to 5 mol %, preferably 0.3 to 3 mol % in the total oil-soluble monomer, because it is advantageous for forming a co-continuous structure.

The surfactant used in step I according to the method for producing the second monolith is, but not particularly limited, capable of forming a water-in-oil (W/O) emulsion when water is mixed with an oil-soluble monomer containing no ion exchange group. Examples thereof include nonionic surfactants such as sorbitan monooleate, sorbitan monolaurate, sorbitan monopalmitate, sorbitan monostearate, sorbitan trioleate, polyoxyethylene nonylphenyl ether, polyoxyethylene stearyl ether, polyoxyethylene sorbitan monooleate; anionic surfactants such as potassium oleate, sodium dodecylbenzenesulfonate, sodium dioctyl sulfosuccinate; cationic surfactants such as distearyl dimethylammonium chloride; and amphoteric surfactants such as lauryldimethylbetaine. These surfactants can be used alone or in combination of two or more thereof. The water-in-oil emulsion is an emulsion in which the oil phase is a continuous phase and water droplets are dispersed therein. The amount of the above-mentioned surfactant added varies greatly depending on the kind of the oil-soluble monomer and the size of the target emulsion particles (macropores), and therefore cannot be unequivocally determined, but it can be selected in the range of about 2 to 70% by mass of the total amount of the oil-soluble monomer and the surfactant.

Further, in the step I according to the method for producing the second monolith, a polymerization initiator may be used as necessary when forming a water-in-oil emulsion. As the polymerization initiator, a compound that generates radicals by heat or light irradiation is preferably used. The polymerization initiator may be water-soluble or oil-soluble, and may be, for example, 2,2′-azobis(isobutyronitrile), 2,2′-azobis(2,4-dimethylvaleronitrile), 2,2′-azobis(2-methylbutyronitrile), 2,2′-azobis(4-methoxy-2,4-dimethyl-valeronitrile), 2,2′-azobis(dimethyl-isobutyrate), 4,4′-azobis(4-cyanovaleric acid), 1,1′-azobis(cyclohexane-1-carbonitrile), benzoyl peroxide, lauroyl peroxide, potassium persulfate, ammonium persulfate, tetramethylthium disulfide, hydrogen peroxide-ferrous chloride, sodium persulfate-sodium acid sulfite and the like.

As a mixing method for forming a water-in-oil emulsion by mixing an oil-soluble monomer containing no ion-exchange group, a surfactant, water and a polymerization initiator in the step I according to the method for producing the second monolith is, but not particularly limited, a method of mixing each component at once, or a method of mixing oil-soluble components, which are an oil-soluble monomer, the surfactant, and an oil-soluble polymerization initiator, and a water-soluble component, which is water or a water-soluble polymerization initiator, are separately dissolved, and then uniformly mixing respective components. The mixing device for forming the emulsion is not particularly limited, and a normal mixer, homogenizer, high-pressure homogenizer, or the like can be used, and an appropriate device may be selected to obtain the desired emulsion particle size. Further, the mixing conditions are not particularly limited, and the stirring rotation speed and the stirring time capable of obtaining the desired emulsion particle size can be arbitrarily set.

The monolith intermediate (2) obtained in the step I according to the method for producing the second monolith is an organic polymer material having a crosslinked structure, preferably an aromatic vinyl polymer. The cross-linking density of the polymer material is not particularly limited, but contains 0.1 to 5 mol %, preferably 0.3 to 3 mol % of cross-linked structural units with respect to all the structural units constituting the polymer material. If the crosslinked structural unit is less than 0.3 mol %, the mechanical strength is insufficient, which is not preferable. On the other hand, if it exceeds 5 mol %, the structure of the monolith tends to deviate from the co-continuous structure, which is not preferable. In particular, when the total pore volume is 16 to 20 ml/g, the crosslinked structural unit is preferably less than 3 mol % in order to form a co-continuous structure.

In the step I according to the method for producing the second monolith, the type of the polymer material of the monolith intermediate may be the same as that of the polymer material of the first monolith.

The total pore volume per weight of the monolith intermediate obtained in step I according to the method for producing the second monolith is more than 16 mL/g and 30 mL/g or less, preferably more than 16 mL/g and 25 mL/g or less. That is, although this monolithic intermediate is basically a continuous macropore structure, the apertures (mesopores), which are the overlapping portions between macropores, are much larger, so the skeleton that makes up the monolithic structure is as close as possible to a one-dimensional rod-like skeleton from a two-dimensional wall. When this is allowed to coexist in the polymerization system, a porous body having a co-continuous structure is formed using the structure of the monolith intermediate as a mold. If the total pore volume is too small, the structure of the monolith obtained after polymerizing the vinyl monomer changes from a co-continuous structure to a continuous macropore structure, which is not preferable. On the other hand, if the total pore volume is too large, When the mechanical strength of the monolith obtained after polymerizing the vinyl monomer is lowered, or if an ion exchange group is introduced, the ion exchange capacity per volume is lowered, which is not preferable. In order to make the total pore volume of the monolith intermediate within the above range, the ratio of the monomer to water may be approximately 1:20 to 1:40.

Further, in the monolith intermediate obtained in the step I according to the method for producing the second monolith, the average diameter of the apertures (mesopores), which are the overlapping portions between macropores, is 5 to 100 μm in a dry state. When the average diameter of the apertures is 5 μm or more in a dry state, it is possible to suppress the opening diameter of the monolith obtained after polymerizing the vinyl monomer from becoming small, and it is possible to suppress the pressure loss during fluid permeation from becoming large. On the other hand, when the average diameter is 100 μm or less, the opening diameter of the monolith obtained after polymerizing the vinyl monomer does not become too large, and the contact between the liquid to be treated and the monolith ion exchanger becomes sufficient, resulting in suppressing a decrease in capture performance. The monolith intermediate preferably has a uniform structure with uniform macropore size and opening diameter, but is not limited to this, and non-uniform macropores larger than the uniform macropore size are scattered in the uniform structure.

The step II of the method for producing the second monolith is a process of preparing a mixture of an aromatic vinyl monomer, a cross-linking agent of 0.3 to 5 mol % in a total oil-soluble monomer having at least two or more vinyl groups in one molecule, an organic solvent which can dissolve the aromatic vinyl monomer and the cross-linking agent, but does not dissolve a polymer generated by polymerizing the aromatic vinyl monomer, and a polymerization initiator. It should be noted that there is no order between the step I and the step II, and the step II may be performed after the step I, or the step I may be performed after the step II.

As the aromatic vinyl monomer used in the step II of the method for producing the second monolith, there is no restriction as long as it is a lipophilic aromatic vinyl monomer containing a polymerizable vinyl group in the molecule and having high solubility in an organic solvent. It is preferable to select a vinyl monomer that produces a polymer material of the same type or similar to the monolith intermediate (2) coexisting in the above-mentioned polymerization system. Specific examples of these vinyl monomers include styrene, α-methylstyrene, vinyltoluene, vinylbenzyl chloride, vinylbiphenyl, vinylnaphthalene and the like. These monomers may be used alone or in combination of two or more. Preferred aromatic vinyl monomers are styrene, vinylbenzyl chloride and the like.

The amount of the aromatic vinyl monomer added in the step II according to the method for producing the second monolith is 5 to 50 times, preferably 5 to 40 times by weight that of the monolith intermediate coexisting at the time of polymerization. When the amount of the aromatic vinyl monomer added is 5 times or more that of the monolith intermediate, the rod-shaped skeleton can be made thicker, and when an ion exchange group is introduced, the ion exchange capacity per volume after the introduction of the ion exchange group can be suppressed to be small. On the other hand, when the amount of the aromatic vinyl monomer added is 50 times or less, the diameter of the continuous pores does not become too small, and it is possible to suppress an increase in pressure loss during liquid passage.

As the crosslinking agent used in the step II of the method for producing the second monolith, a crosslinking agent containing at least two polymerizable vinyl groups in the molecule and having high solubility in an organic solvent is preferably used. Specific examples of the crosslinking agent include divinylbenzene, divinylnaphthalene, divinylbiphenyl, ethylene glycol dimethacrylate, trimethylolpropane triacrylate, butanediol diacrylate and the like. These crosslinking agents can be used alone or in combination of two or more. Preferred crosslinking agents are aromatic polyvinyl compounds such as divinylbenzene, divinylnaphthalene and divinylbiphenyl because of their high mechanical strength and stability against hydrolysis. The amount of the crosslinking agent used is 0.3 to 5 mol %, particularly 0.3 to 3 mol % with respect to the total amount (total oil-soluble monomers) of the vinyl monomer and the crosslinking agent. When the amount of the crosslinking agent used is 0.3 mol % or more, the mechanical strength of the monolith is not insufficient, while when the ion exchange group is introduced at 5 mol % or less, it is not difficult to quantitatively introduce the ion exchange group. The amount of the cross-linking agent used is preferably substantially equal to the crosslinking density of the monolith intermediate coexisting during the polymerization of the vinyl monomer/crosslinking agent. If the amounts used for both are too far apart, the crosslink density distribution will be biased in the produced monolith, and when an ion exchange group is introduced, cracks are likely to occur during the ion exchange group introduction reaction.

The organic solvent used in the step II of the method for producing the second monolith is an organic solvent in which the aromatic vinyl monomer and the crosslinking agent are dissolved but the polymer produced by the polymerization of the aromatic vinyl monomer is not dissolved, in other words, it is a poor solvent for the polymer produced by polymerizing the aromatic vinyl monomer. Since the organic solvent varies greatly depending on the kinds of aromatic vinyl monomers, it is difficult to list general specific examples. However, for example, when the aromatic vinyl monomer is styrene, examples of the organic solvent include alcohols such as methanol, ethanol, and propanol, butanol, hexanol, cyclohexanol, octanol, 2-ethylhexanol, decanol, dodecanol, propylene glycol and tetramethylene glycol; linear (poly)ethers such as diethyl ether, butyl cellosolve, polyethylene glycol, polypropylene glycol, and polytetramethylene glycol; linear saturated hydrocarbons such as hexane, heptane, octane, isooctane, decane and dodecane; esters such as ethyl acetate, isopropyl acetate, cellosolve acetate and ethyl propionate. Further, even good solvents of polystyrene, such as dioxane, THF, and toluene, can be used together with the above-mentioned poor solvents, if they are used in small amounts as an organic solvent. The amount of these organic solvents used is preferably such that the concentration of the aromatic vinyl monomer is 30 to 80% by mass. When the amount of the organic solvent is an amount that results in 30% by mass or more of the concentration of the aromatic vinyl monomer, it is possible to suppress a decrease in the polymerization rate or deviation of the monolith structure after polymerization from the range of the second monolith when deviating the above range. On the other hand, when the concentration of the aromatic vinyl monomer is 80% by mass or less, the runaway of polymerization can be suppressed.

As the polymerization initiator used in the step II of the method for producing the second monolith, a compound that generates radicals by heat or light irradiation is preferably used. The polymerization initiator is preferably oil-soluble. Specific examples of the polymerization initiator include 2,2′-azobis(isobutyronitrile), 2,2′-azobis(2,4-dimethylvaleronitrile), 2,2′-azobis(2-methylbutyronitrile), 2,2′-azobis(4-methoxy-2,4-dimethylvaleronitrile), 2,2′-azobis(isobutyrate)dimethyl, 4,4′-azobis(4-cyanovaleric acid), 1,1′-azobis(cyclohexane-1-carbonitrile), benzoyl peroxide, lauroyl peroxide, potassium persulfate, ammonium persulfate, and tetramethylthiuram disulfide. The amount of the polymerization initiator used varies greatly depending on the kinds of the monomers, polymerization temperatures, etc., but can be used in the range of about 0.01 to 5% by mass with respect to the total mass of the vinyl monomer and the crosslinking agent.

The step III according to the method for producing the second monolith is a step in which the mixture obtained in step II is polymerized under static conditions and in the presence of the monolith intermediate obtained in step I to change the continuous macropore structure of the monolith intermediate into co-continuous structure to obtain the second monolith that is a co-continuous structure monolith. The monolith intermediate used in step III plays an extremely important role in creating a monolith having the structure defined in the present invention. As disclosed in JP07-501140A, when a vinyl monomer and a crosslinking agent are statically polymerized in a specific organic solvent in the absence of a monolithic intermediate, a particle-aggregating monolithic organic porous material is obtained. In contrast, when a monolith intermediate having a specific continuous macropore structure is present in the polymerization system like the second monolith, the structure of the monolith after the polymerization changes dramatically, the particle aggregation structure disappears, and the above-mentioned second monolith with a co-continuous structure of is obtained. Although the reason for this has not been clarified in detail, in the absence of the monolith intermediate, the crosslinked polymer produced by the polymerization separates and precipitates in the form of particles to form a particle agglomerate structure, whereas a porous body (intermediate) having a large total pore volume is present in the polymerization system, the vinyl monomer and the crosslinking agent are adsorbed or distributed from the liquid phase to the skeleton of the porous body, and polymerization proceeds in the porous body. It is considered that the skeleton constituting the monolith structure changes from a two-dimensional wall surface to a one-dimensional rod-shaped skeleton to form a second monolith having a co-continuous structure.

In the method for producing the second monolith, the internal volume of the reaction vessel is not particularly limited as long as it has a size that allows the monolith intermediate to exist in the reaction vessel. The reaction vessel may have a gap around the monoliths in a plan view when the monolith intermediate is placed in the reaction vessel, or may be that a monolith intermediate can be inserted into the reaction vessel without a gap. Of these, the one, in which the monolith with a thick skeleton after polymerization does not receive pressure from the inner wall of the vessel and enters the reaction vessel without a gap, is more efficient because there is no distortion of the monolith and no waste of reaction materials, etc. Even if the internal volume of the reaction vessel is large and there is a gap around the monolith after polymerization, the vinyl monomer and the crosslinking agent are adsorbed and distributed to the monolith intermediate, so that no particle aggregation structure is formed in the gap portion of the reaction vessel.

In the step III of the method for producing the second monolith, the monolith intermediate is placed in a reaction vessel in a state of being immersed in the mixture (solution). As described above, the mixing ratio of the mixture obtained in the step II to the monolith intermediate is suitable such that the amount of the vinyl monomer added is 3 to 50 times, preferably 4 to 40 times, by weight, with respect to the monolith intermediate. This makes it possible to obtain a second monolith having a thick skeleton while having an appropriate opening diameter. In the reaction vessel, the vinyl monomer and the crosslinking agent in the mixture are adsorbed and distributed onto the skeleton of the static monolith intermediate, and the polymerization proceeds in the skeleton of the monolith intermediate. A second monolith with a co-continuous structure, in which pores of moderate size are three-dimensionally continuous and thick skeletons are three-dimensionally continuous, can be obtained.

Various conditions are selected for the polymerization conditions of the step III according to the method for producing the second monolith, depending on the kinds of the monomer and the kinds of the initiator. For example, when 2,2′-azobis(isobutyronitrile), 2,2′-azobis(2,4-dimethyl-valeronitrile), benzoyl peroxide, lauroyl peroxide, potassium persulfate, etc. are used as the initiator, in a sealed vessel under an inert atmosphere, the polymerization may be carried out by heating at 30 to 100° C. for 1 to 48 hours. By heat polymerization, the vinyl monomer and the crosslinking agent adsorbed and distributed on the skeleton of the monolith intermediate polymerize in the skeleton so as to thicken the skeleton. After completion of the polymerization, the contents are taken out and extracted with a solvent such as acetone for the purpose of removing the unreacted vinyl monomer and the organic solvent to obtain a second monolith.

The second monolith ion exchanger is obtained by performing step IV of introducing an ion exchange group into the second monolith obtained in step III.

The method of introducing an ion exchange group into the second monolith is the same as the method of introducing an ion exchange group into the first monolith.

The second monolith and the second monolith ion exchanger have high mechanical strength because they have a thick skeleton even though the size of the three-dimensionally continuous pores is remarkably large. Further, since the second monolith ion exchanger has a thick skeleton, the ion exchange capacity per volume in a water-wet state can be increased, and the liquid to be treated can be passed through at a low pressure and a large flow rate for a long period of time.

Compared to the porous membranes and ion exchange resins used in other methods of analyzing ionic impurity content, the monolith ion exchanger allows the captured ionic impurity elements to be more easily eluted by the eluent, so the analysis method of the present invention can lower the acid concentration of the eluent and thus the lower limit of quantitation.

Compared to the porous membranes and ion exchange resins used in other methods of analyzing ionic impurity content, the monolith ion exchanger allows the captured metal element is more easily eluted by the eluent, so the analysis method of the present invention can shorten the analysis time because the time required for the elution step is shortened.

Compared to the porous membranes and ion exchange resins used in other methods of analyzing ionic impurity content, the monolith ion exchanger allows an increase of the flow rate of the water to be analyzed, so the analysis method of the present invention can shorten the analysis time because the time required for the liquid passing step is shortened.

Conventionally, when the metal impurity content in the water to be analyzed is very low, for example, when it is 1 ppt or less, it is necessary to pass a large volume of the water to be analyzed through the adsorbent. In although the analysis method of the present invention, the metal impurities in the water to be analyzed (ultra-pure water) are very low, less than 1 ng/L, the captured metal impurity element can be easily eluted by the eluent, since the volume of the porous ion exchanger per unit is 0.5 to 5.0 ml, and the differential pressure coefficient is 0.01 MPa/LV/m or less. Therefore, the amount of the eluent used can be reduced, and the volume of ultrapure water passed through the porous (monolith) ion exchanger can be reduced. The volume of nitric acid or hydrochloric acid used in the elution step needs to be at least 10 times the volume as described in WO 2019/221186A1. In addition, the minimum volume of eluent required for analysis without contamination with an analytical instrument is 5 ml. The volume of the eluent is preferably 50 ml at the maximum in order to reduce the concentrated amount needed to analyze until a low concentration. From this, it is desirable that the volume of the monolith exchanger required per unit is 0.5 to 5.0 ml. The differential pressure coefficient of the ion exchanger is 0.01 MPa/LV/m or less, preferably 0.005 MPa/LV/m or less. Further, since the flow rate of ultrapure water can be increased, a large amount of liquid can be passed in a short time, so that the time required for the capturing step in the analysis can be very shortened. Further, in this case, the pressure coefficient in the capturing step of the analysis method of the present invention is preferably 0.1 to 10.0 L/min./MPa, particularly preferably 2.0 to 10.0 L/min./MPa.

The measurement apparatus (metal impurity capturing device) of the first embodiment of the present invention is a measurement apparatus for measuring the content of metal impurities in a liquid, and includes:
an ion exchanger through which the liquid is passed and
an integrated flow meter for measuring the amount of liquid passed through the ion exchanger,
characterized in that the ion exchanger is provided by connecting two or more units of the same ion type ion exchangers in series, the volume of the ion exchanger per unit is 0.5 to 5.0 ml, and the differential pressure coefficient per unit is 0.01 MPa/LV/m or less.

The size of the container used for the flow cell is not particularly limited, but it is desirable to set it according to the size of the ion exchanger of the above volume to be filled. If the cross-sectional area of the container to be filled is too small, the pressure loss will be large and it will take time to concentrate. If the cross-sectional area is too large, the length of the exchanger will be short and ions will not be captured and correct analysis will not be possible. Therefore, it is desirable that the diameter of the cross section is ϕ0.2 to 5 cm. The shape of the container is not particularly limited, but a shape that can reduce short paths such as a columnar shape is desirable.

The measurement apparatus of the present invention can have various forms shown in FIGS. 1 to 3.

The integrated flow meter according to the measurement apparatus of the present invention is not particularly limited as long as it can measure and integrate the volume of liquid to be introduced.

The measurement apparatus of the present invention can include a supply pipe for supplying the liquid to be analyzed and the eluent to the monolith ion exchanger in the flow cell, an introduction pipe for introducing the discharge liquid discharged from the porous ion exchanger into the integrated flow meter, and a discharge pipe for discharging the discharge liquid discharged from the integrated flow meter to the outside of the apparatus. Further, to control the flow rate, a valve may be provided between the flow cell and the integrated flow meter or immediately after the integrated flow meter.

It is preferable that the measurement apparatus of the present invention is provided with a sealing means for sealing the inside so that impurities are not mixed into the inside after the apparatus is removed from the tube to which the liquid to be analyzed is supplied.

As the ion exchanger according to the measurement apparatus of the present invention, the above-mentioned monolith ion exchanger can be used.

### EXAMPLES

Next, the present invention will be specifically described with reference to examples, but this is merely an example and does not limit the present invention.

A second cationic monolith ion exchanger was produced in the same manner as in Reference Example 17 of the examples of the specification according to JP2010-234357A.

### Reference Example 1

### <Production of Cationic Monolith Ion Exchanger>

(Step I; Production of Monolith Intermediate) 5.4 g of styrene, 0.17 g of divinylbenzene, 1.4 g of sorbitan monooleate (hereinafter abbreviated as SMO) and 0.26 g of 2,2′-azobis (isobutyronitrile) were mixed and uniformly dissolved. Next, the styrene/divinylbenzene/SMO/2,2′-azobis (isobutyronitrile) mixture was added to 180 g of pure water, and was stirred by using a vacuum stirring defoaming mixer (manufactured by EME), which is a planetary stirring device, under reduced pressure in a temperature range of 5 to 20° C. to obtain a water-in-oil emulsion. This emulsion was immediately transferred to a reaction vessel, sealed, and polymerized at 60° C. for 24 hours under static condition. After completion of the polymerization, the contents were taken out, extracted with methanol, and dried under reduced pressure to produce a monolith intermediate having a continuous macropore structure. When the internal structure of the monolith intermediate (dried material) thus obtained was observed by SEM images, the wall portion separating the two adjacent macropores was extremely thin and rod-shaped, but had a continuous cell structure. The average diameter of the opening (mesopore) at the portion where the macropore and the macropore overlap was 70 μm and the total pore volume was 21.0 ml/g, which was measured by the mercury intrusion method.

### (Manufacturing of Co-Continuous Structure Monolith)

Next, 76.0 g of styrene, 4.0 g of divinylbenzene, 120 g of 1-decanol, and 0.8 g of 2,2′-azobis(2,4-dimethylvaleronitrile) were mixed and uniformly dissolved (step II). Next, the above monolith intermediate was cut into a disk shape having a diameter of 70 mm and a thickness of about 40 mm, and 4.1 g was separated. The separated monolith intermediates were placed in a reaction vessel having an inner diameter of 110 mm, immersed in the styrene/divinylbenzene/1-decanol/2,2′-azobis(2,4-dimethylvaleronitrile) mixture, deformed in a reduced pressure chamber, and then the reaction vessel was sealed and polymerized at 60° C. for 24 hours under static condition. After completion of the polymerization, a monolithic content having a thickness of about 60 mm was taken out, Soxhlet-extracted with acetone, and then dried under reduced pressure at 85° C. overnight (step III).

When the internal structure of the monolith (dried product) containing 3.2 mol % of the crosslinked component composed of the styrene/divinyl-benzene-copolymer thus obtained was observed by SEM, the monolith had a co-continuous structure that skeleton and pores are three-dimensionally continuous, respectively, and both phases were intertwined. The thickness of the skeleton measured from the SEM image was 17 μm. The size of the three-dimensionally continuous pores of the monolith measured by the mercury intrusion method was 41 μm, and the total pore volume was 2.9 ml/g.

### (Manufacturing of a Cationic Monolith Ion Exchanger (CEM) Having a Co-Continuous Structure)

The monolith produced by the above method was cut into a cylinder having a diameter of 75 mm and a thickness of about 15 mm. The weight of the monolith was 18 g. 1500 ml of dichloromethane was added thereto, and the mixture was heated at 35° C. for 1 hour, cooled to 10° C. or lower, 99 g of chlorosulfuric acid was gradually added, the temperature was raised, and the reaction was carried out at 35° C. for 24 hours. Then, methanol was added, and the remaining chlorosulfuric acid was quenched, washed with methanol to remove dichloromethane, and further washed with pure water to obtain a cationic monolith ion exchanger CEM having a co-continuous structure.

### (Analysis of Cationic Monolith Ion Exchanger CEM)

Further, when a part of the obtained cationic monolith ion exchanger was cut out and dried, and its internal structure was observed by SEM, it was confirmed that the monolith ion exchanger maintained co-continuous structure. The swelling rate of the monolith ion exchanger before and after the reaction was 1.4 times, and the cation exchange capacity per volume was 0.72 mg-equivalent/ml in a water-wet state. The size of the continuous pores of the monolith in the water-wet state was estimated from the value of the monolith and the swelling rate of the cation exchanger in the water-wet state to be 70 μm, the diameter of the skeleton was 23 μm, and the total pore volume was 2.9 ml/g.

The differential pressure coefficient, which is an index of the pressure loss when water has been permeated, was 0.005 MPa/m·LV. Further, when an ion exchange band length with respect to sodium ion of the monolith ion exchanger was measured, the ion exchange band length at LV=20 m/h was 16 mm, it was not only significantly shorter than the value (320) of Amberlite IR120B (product name, manufactured by Roam and Haas), a commercially available strongly acidic cation exchange resin, but also shorter than the value of conventional cationic monolith ion exchangers having an open cell structure.

Next, in order to confirm the distribution state of the sulfonic acid group in the monolith ion exchanger, the distribution state of the sulfur atom was observed by EPMA. As a result, it was observed that the sulfonic acid group was uniformly introduced into the skeleton surface and the inside of the skeleton (cross-sectional direction) of the monolith ion exchanger.

### Comparative Example 1

The above cationic monolith ion exchanger was cut into a shape of 10 mm in diameter×50 mm in height (2.87 mL) and placed in a packed container made of PFA (tetrafluoroethylene-perfluoroalkyl vinyl ether copolymer).

Next, into the packed container, ultrapure water was passed at about 500 mL/min (SV=8000 h−1, LV=400 m/h), so that the concentration amount became 5000 L, and a liquid passing was performed to one unit of the cationic monolith ion exchanger (CEM1).

Next, 2N nitric acid was used as an eluent, and the eluent was recovered at a volume of 50 mL. The recovered eluent was measured by ICP-MS, and the concentration of each metal element shown in Table 1 was measured.

### (Analysis)

The content of each element captured by the cationic monolith ion exchanger was measured by ICP-MS (manufactured by Agilent Technologies, Model 8900).

In the analysis of the content by ICP-MS, a calibration curve of the count value (CPS) and the metal content was prepared in advance using a standard sample of a plurality of contents, and a test sample (test water or treated water) was measured, and the metal content corresponding to the count value was defined as the metal content of the test water or the treated water based on the calibration curve.

### Example 1

The ultrapure water passing step, elution step and analysis step were carried out in the same manner as in Comparative Example 1 except that two units (CEM1 and CEM2) of the flow cells of the cationic monolith ion exchanger were connected in series. The results are shown in Table 1.

### Example 2

The ultrapure water passing step, elution step and analysis step were carried out in the same manner as in Comparative Example 1 except that 3 units (CEM1, CEM2, CEM3) of the flow cells of the cationic monolith ion exchanger were connected in series. The results are shown in Table 1.

TABLE 1

| | | Element Amount [pg/L] | | | |
|---|---|---|---|---|---|
| | Exchanger | Na | Mg | Ca | Fe |
| Comparative Example 1 | CEM1 | 119 | 10 | 86 | 11 |
| Example 1 | CEM1 | 119 | 10 | 86 | 11 |
| | CEM2 | 5 | <1 | 8 | 1 |
| Example 2 | CEM1 | 119 | 10 | 86 | 11 |
| | CEM2 | 5 | <1 | 8 | 1 |
| | CEM3 | <1 | <1 | <1 | <1 |

In the table, “<1 [pg/L]” indicates that it is less than the lower limit of quantification of this method. Therefore, regarding Mg, it was confirmed from Comparative Example 1, Example 1, and Example 2 that the concentration in ultrapure water was 10 μg/L, but for other elements, it was confirmed that one unit of monolith ion exchanger could not sufficiently capture and did not show the correct metal concentration in ultrapure water from the results of Examples 1 and 2. As shown in Example 2, it was confirmed that CEM3 was below the lower limit of quantification for all metal elements, and the concentration of CEM1+CEM2 was the metal concentration in ultrapure water.

The concentration is calculated by the following formula (1).

$$\begin{matrix}[ Math . 1 ] & \\ \begin{matrix}Concentration \\ [ ng / L to pg / L ]\end{matrix} = \frac{\begin{matrix}Eluent Volume \\ [ L ]\end{matrix} \times \begin{matrix}Concentrated Concentration \\ ( ICP - MS ) [ ng / L ]\end{matrix}}{Concentrated Volume [ L ]} & ( 1 )\end{matrix}$$

While the limit of the conventional method (heat concentration method) is 0.1 ng/L, the lower limit of quantification of 1 μg/L (0.001 ng/L) can be analyzed by the adsorption and concentration method of the present invention.

### (Manufacturing of Anionic Monolith Ion Exchanger)

The monolith produced by the above method was cut into a disk shape having an outer diameter of 70 mm and a thickness of about 15 mm. To this, 1400 ml of dimethoxymethane and 20 ml of tin tetrachloride were added, and 560 ml of chlorosulfuric acid was added dropwise under ice-cooling. After completion of the dropping, the temperature was raised and the reaction was carried out at 35° C. for 5 hours to introduce a chloromethyl group into the monolith. After completion of the reaction, the mother liquor was siphoned out and washed with a mixed solvent of THF/water=2/1, and further washed with THF. To this chloromethylated monolith organic porous material, 1000 ml of THF and 600 ml of a 30% aqueous solution of trimethylamine were added, and the mixture was reacted at 60° C. for 6 hours. After completion of the reaction, the product was washed with a mixed solvent of methanol/water, followed by washing with pure water to isolate an anionic monolith ion exchanger.

### Comparative Example 2

The anionic monolith ion exchanger is cut into a shape having a diameter of 10 mm and a height of 50 mm, and filled in a packed container made of PFA (tetrafluoroethylene/perfluoroalkyl vinyl ether copolymer) to form a flow cell of an anionic monolith ion exchanger.

Next, ultrapure water was passed through the packed container at about 100 mL/min. (SV=1600 h⁻¹, LV=80 m/h), so that the volume of concentration was 100 L to one unit of the anionic monolith ion exchanger (AEM1).

Next, 2N nitric acid was used as the eluent, and the eluent was recovered at a volume of 50 mL. The recovered eluent was measured by ICP-MS to determine the concentration of the boron element shown in Table 2.

### (Analysis)

The content of each element captured by the monolith ion exchanger was measured by ICP-MS (manufactured by Agilent Technologies, Model 8900).

In the analysis of the content by ICP-MS, a calibration curve of the count value (CPS) and the metal content is prepared in advance using a standard sample of a plurality of contents, and a test sample (test water or treated water) was measured, and the metal content corresponding to the count value was defined as the metal content of the test water or the treated water based on the calibration curve.

### Example 3

The ultrapure water passing step, elution step and analysis step were carried out in the same manner as in Comparative Example 2 except that two units (AEM1 and AEM2) of the flow cells of the anionic monolith ion exchanger were connected in series. The results are shown in Table 2.

### Example 4

Ultrapure water passing step, elution step and analysis step were carried out in the same manner as in Comparative Example 1 except that 3 units (AEM1, AEM2, AEM3) of the flow cells of the anionic monolith ion exchanger were connected in series. The results are shown in Table 2.

TABLE 2

| | | Boron concentration |
|---|---|---|
| | Exchanger | [ng/L] |
| Comparative Example 2 | AEM1 | 0.22 |
| Example 3 | AEM1 | 0.22 |
| | AEM2 | 0.15 |
| Example 4 | AEM1 | 0.22 |
| | AEM2 | 0.15 |
| | AEM3 | <0.05 |

In Table 2, “<0.05 [ng/L]” indicates that it is less than the lower limit of quantification of this method.

As shown in Table 2, in Comparative Example 2, it was confirmed that the boron concentration in ultrapure water was 0.22 ng/L, but from the results of Examples 3 and 4, it was confirmed that one unit of anionic monolith ion exchanger could not sufficiently capture and did not show the correct boron concentration in ultrapure water. As shown in Example 4, it was confirmed that AEM3 was below the lower limit of quantification and the concentration of AEM1+AEM2 was 0.37 ng/L, which was the boron concentration in ultrapure water. As described above, the number of units connected in series of the ion exchanger is preferably the minimum number at which the content of the impurity component in the eluent from the most downstream ion exchanger is less than the lower limit of quantification.

### EXPLANATION OF SIGNS

11 Transfer pipe for transferring ultrapure water
12 Discharge pipe for water to be analyzed
13 Flow cell
13A, 13A′, 13B, 13B′ Flow cell
14, 14′ Integrated flow meter
15, 15′ Measurement apparatus
15 16 First branch pipe
16′ Second branch pipe
CEM Cationic monolith ion exchanger
AEM Anionic monolith ion exchanger
UPM Ultrapure water

1. A method for analyzing a content of a metal impurity in a liquid, which comprises:
passing the liquid through an ion exchanger;
eluting and recovering the metal impurity captured in the ion exchanger with an eluent; and
analyzing the eluent containing the eluted metal impurity and measuring the content of the metal impurity in the eluent,
wherein the ion exchanger is used by connecting two or more units of the same ion-type ion exchanger in series,
the volume of the ion exchanger per unit is 0.5 to 5.0 ml, and the differential pressure coefficient per unit is 0.01 MPa/LV/m or less.

2. The method for analyzing according to claim 1, wherein the eluting and the measuring are performed for each unit of the ion exchanger in order from the top to bottom, and when the content of the metal impurity in the liquid measured becomes less than the lower limit of quantification, the total amount of metal impurities in the liquid until it becomes less than the lower limit of quantification is defined as the amount of metal impurities in the liquid.

3. The method for analyzing according to claim 1, wherein the ion exchanger is a monolithic organic porous ion exchanger.

4. The method for analyzing according to claim 1, wherein the number of units of the ion exchanger is the minimum number for which the content of metal impurity analyzed based on the last ion exchanger is less than the lower limit of quantification.

5. A measurement apparatus for measuring a content of a metal impurity in a liquid comprising:
an ion exchanger through which the liquid is passed, and
an integrated flowmeter for measuring the volume of liquid passed through the ion exchanger,
wherein the ion exchanger is used by connecting two or more units of the same ion-type ion exchanger in series,
the volume of the ion exchanger per unit is 0.5 to 5.0 ml, and the differential pressure coefficient per unit is 0.01 MPa/LV/m or less.

6. The measurement apparatus according to claim 5, wherein the ion exchanger is a monolithic organic porous ion exchanger.

7. The measurement apparatus according to claim 5, wherein the number of units of the ion exchanger is the minimum number for which the content of metal impurity analyzed based on the last ion exchanger is less than the lower limit of quantification.

8. The method for analyzing according to claim 2, wherein the ion exchanger is a monolithic organic porous ion exchanger.

9. The method for analyzing according to claim 2, wherein the number of units of the ion exchanger is the minimum number for which the content of metal impurity analyzed based on the last ion exchanger is less than the lower limit of quantification.

10. The method for analyzing according to claim 3, wherein the number of units of the ion exchanger is the minimum number for which the content of metal impurity analyzed based on the last ion exchanger is less than the lower limit of quantification.

11. The method for analyzing according to claim 8, wherein the number of units of the ion exchanger is the minimum number for which the content of metal impurity analyzed based on the last ion exchanger is less than the lower limit of quantification.

12. The measurement apparatus according to claim 6, wherein the number of units of the ion exchanger is the minimum number for which the content of metal impurity analyzed based on the last ion exchanger is less than the lower limit of quantification.
